(19) United States
(12) Patent Application Publication
(10) Pub. No.: US 2022/0378888 A1
(43) Pub. Date: Dec. 1, 2022

(54) SCALABLE AND FACILE CELL-MEMBRANE-COATING TECHNOLOGY FOR BOTH POSITIVELY AND NEGATIVELY CHARGED PARTICLES

(71) Applicant: THE TRUSTEES OF COLUMBIA UNIVERSITY IN THE CITY OF NEW YORK, New York, NY (US)

(72) Inventors: Kam W. LEONG, New York, NY (US); Hanze HU, New York, NY (US); Dan SHAO, New York, NY (US); Chao YANG, New York, NY (US)

(21) Appl. No.: 17/887,341

(22) Filed: Aug. 12, 2022

Related U.S. Application Data
(63) Continuation of application No. PCT/US21/17823, filed on Feb. 12, 2021.
(60) Provisional application No. 62/976,865, filed on Feb. 14, 2020.

(51) Int. Cl. A61K 39/00; B01J 13/10; A61K 39/39; A61P 35/00

(52) U.S. Cl. CPC A61K 39/0011; B01J 13/10; A61K 39/39; A61P 35/00; A61K 2039/60

(57) ABSTRACT

A method for synthesizing cell membrane-biomimetic nanotherapeutics can include coating core particles with cell membrane materials using flash nanocomplexation (FNC). FNC is a turbulent mixing and self-assembly method that can produce cell membrane-coated nanotherapeutics in a reproducible and scalable manner. The FNC-produced cell membrane-coated particles demonstrate lower aggregation, polydispersity, and zeta potential, than nanoparticles prepared by conventional coating methods, such as conventional bulk-sonication. As such, the present method achieves more complete, homogeneous and controllable coating than conventional bulk-sonication methods.

### CROSS-REFERENCE

The present application is a continuation of PCT/US2021/017823, filed on Feb. 12, 2021, which application claims priority to provisional U.S. Patent Application No. 62/976,865, filed Feb. 14, 2020, which was filed by the inventors hereof and is incorporated herein by reference in its entirety.

### FIELD

The present subject matter relates to a method for synthesizing cell membrane-biomimetic therapeutics, and particularly, to a method for synthesizing cell membrane-biomimetic therapeutics using flash nanocomplexation.

### BACKGROUND

In recent decades, nanomaterial research has placed significant focus on biomimetics. Biomimetic strategies are useful for designing therapeutic delivery systems that can negotiate biological barriers. Size reduction, colloidal stability, particle protection, and enhanced permeability and retention (EPR) properties in one or more dimensions are practical considerations in preparing nanoparticles for efficient diagnostic and therapeutic applications. The clinical and biological complexity of maladies has contributed to the advent of biomimetic nanocarriers as an alternative approach to conventional nanoparticles by exploiting cell-like functions.

Cell membrane-coating of therapeutic nanoparticles is a promising biomimetic strategy. Cell membrane coating technology integrates the biological features of cell membranes with the functional versatility of nanomaterials. Production involves coating synthetic nanoparticle backbone materials with a naturally-derived cell membrane layer to form a biomimicking ensemble. These nanotherapeutics have shown advantageous physical properties such as improved stability and longer circulation times, and intrinsic functionalities inherited from the donor cell source such as toxin neutralization, homologous targeting, and immune invasion. However, producing regulatory agency-approved cell membrane-coated nanomaterials requires a high level of manufacturing sophistication. Conventional approaches to fabricating cell membrane-coated nanomaterials rely on two main strategies: extrusion and sonication. Extrusion produces homogeneous coatings and uniform size, but is prohibitively time-consuming; sonication offers a facile approach to produce sufficient product, but quality is compromised in several ways. Further, conventional coating methods have not been able to achieve a standardizable, batch-to-batch-consistent, and scalable approach for production of such nanoparticles.

Only a modicum of biomimetic nanoparticles has been translated to clinics due to unscalable preparation and batch-to-batch variation. Cell membrane extraction and preservation, cell membrane cloaking through sonication and manual membrane extrusion typically take a considerable amount of time and energy to accomplish. Minimizing batch-to-batch variations from countless benchtops has proven to be even more challenging. Further, the conventional membrane cloaking method is heavily dependent on the mastery of distinctive operators. As such, a facile and robust way to prepare a cell membrane cloaked nanoproduct is essential to popularize and standardize the membrane coating technique and to overcome the translational barriers in nanomedicine.

Accordingly, an efficient and reliable cell membrane coating process that produces biomimetic materials in a timely manner with minimal batch-to-batch variation and complies with good manufacturing practice (GMP) is needed.

### SUMMARY

A method for synthesizing a cell membrane-loaded particle can include coating core particles with cell membrane materials using flash nanocomplexation (FNC). FNC is a turbulent mixing and self-assembly method that can produce cell membrane-coated particles in a reproducible and scalable manner. The FNC-produced cell membrane-coated particles demonstrate lower aggregation, polydispersity, and zeta potential, than particles prepared by conventional coating methods, such as conventional bulk-sonication. The present method achieves more complete and homogeneous coating than conventional bulk-sonication methods.

Importantly, FNC cell membrane coating is effective even on cationic particles, which cannot be achieved using sonication methods. Further, compared with sonication-coated nanovaccines, the FNC-fabricated nanovaccines demonstrate better performance on lymph node targeting, DC antigen presentation, T cell immune-activation, and prophylactic and therapeutic efficacy in melanoma when combined with anti-CTLA-4. Accordingly, FNC represents a universal, robust, and scalable tool that can be used for the manufacturing of cell membrane-based biomimetic nanomedicine.

In an embodiment, a method of using flash nanocomplexation to prepare a cell membrane-loaded particle can include loading a cell membrane material and a core particle into a confined mixing cavity and turbulent mixing of the cell membrane material and the core particle in the mixing cavity to provide the cell membrane-cloaked particles. In an embodiment, the turbulent mixing achieves a turbulent intershearing flow in the confined cavity. In an embodiment, the confined cavity includes a multi-inlet vortex mixer. In an embodiment, a flow rate in the multi-inlet vortex mixer ranges from about 5 mL/min to about 40 mL/min.

### BRIEF DESCRIPTION OF DRAWINGS

Various embodiments will now be described in detail with reference to the accompanying drawings.

FIGS. 1A-1F depict A) a schematic illustration of FNC cell membrane coating; B) a comparison of FNC and bulk sonication methods on PDI and stability of membrane-coated nanoparticles; C) characterization of membrane-coated MSNs using different membrane-to-MSN ratios in terms of size and PDI; D) characterization of membrane-coated MSNs using different membrane-to-MSN ratios in terms of size Zeta potential; E) images of multi-inlet vortex mixer (MIVM) and vials containing total of 40 mL B16-F10 membrane-coated MSNs at 0.5 mg/mL as well as their lyophilized product; F) Total flow rate and production rate for cell membrane-coated MSN using FNC at different Reynolds numbers.

FIG. 2 depicts SEM images of bare cores and cell membrane-coated particles produced using FNC.

FIG. 3 depicts TEM images of bare cores and cell membrane-coated particles produced using FNC FIGS. 4A-4D depict A) size and PDI of raw membrane-coated MSN—Se—Se NPs; B) size and PDI of of raw membrane-coated MSN—Se—Se—NH₂ NPs; C) size and Zeta of raw membrane-coated MSN-Se—Se NPs; and D) size and Zeta potential of raw membrane-coated MSN-Se—Se—NH₂ NPs, produced using bulk sonication or FNC methods. Data represent mean±SD (n=3).

FIGS. 5A-5D depict A) size and PDI of MCF membrane-coated PLGA NPs; B) size and PDI of HepG2 membrane-coated PEI-plasmid NPs, produced by bulk sonication or FNC methods; C) size and Zeta potential of MCF membrane-coated PLGA NPs; and D) Zeta potential of HepG2 membrane-coated PEI-plasmid NPs, produced by bulk sonication or FNC methods. Data represent mean±SD (n=3).

FIGS. 6A-6B depict time-dependent colloidal stability of A) negatively-charged MSNs@RAW, and B) positively-charged MSNs-NH₂@RAW in 10% FBS-containing medium. Data represent mean±SD (n=3).

FIGS. 7A-7F depict A) a schematic illustration of B16-F10 cancer cell membrane-coated, CpG-loaded MSNs (MSN-CpG@CM) produced by FNC; B) TEM images of MSN-CpG, bulk MSN-CpG@CM, and FNC MSN-CpG@CM; C) Gp100 expression on MSN-CpG@CM; D) Size and PDI of MSN-CpG@CM; E) Zeta potential of of MSN-CpG@CM; and F) Long-term stability of MSN-CpG@CM. Data represent mean±SD (n=3) for panels D-F.

FIG. 8 depicts SDS-PAGE protein analysis of MSN-CpG@CM produced using bulk sonication or FNC methods.

FIG. 9 depicts CpG release behavior of MSN-CpG@CM produced using FNC in 1×PBS or 5×110-3 M GSH or 1×10-4 M H₂O₂ for 48 h.

FIGS. 10A-10B depict cell viability of A) BMDCs, and B) RAW 264.7 cells incubated with various concentrations of MSN, MSN-CpG, or MSN-CpG@CM produced by bulk sonication or FNC methods for 24 h. Data represent mean±SD (n=3).

FIGS. 11A-11E depict A) Intracellular colocalization of DiD-labeled B16-F10 membrane modifications and FITC-labeled CpG-loaded MSNs in bone marrow-derived dendritic cells (BMDCs) after incubation for 3 h. Scale bars, 10 m; B) Relative fluorescence intensity of BMDCs after incubation with MSN-CpG@CM for 3 h. Data represent mean±SD (n=3, *p<0.05 vs. MSN-CpG group); C) Fluorescence imaging of popliteal lymph node at indicated time points after footpad injection of free CpG, naked MSN-CpG, or MSN-CpG@CM produced using bulk sonication or FNC methods; D) Quantitation of fluorescence intensity from Cy5.5-labeled CpG in the popliteal lymph node; and E) Uptake of Cy5.5-labeled MSN-CpG@CM by DCs and macrophages in the lymph node at 24 h after injection. Data represent mean±SD (n=3, *p<0.05 vs. CpG group, ^{#}p<0.05 vs. MSN-CpG group, ^{&}p<0.05 vs. bulk MSN-CpG@CM group).

FIGS. 12A-12B depict A) Fluorescence imaging of popliteal lymph node at indicated time points after footpad injection of MSN-CpG@CM produced using bulk sonication or FNC methods; and B) Quantitative fluorescence intensity of DiD-labeled membranes in lymph node. Data represent mean±SD (n=3).

FIGS. 13A-13D relate to APCs were incubated with nanovaccines or various control formulations and depict A) Quantification of DC maturation markers CD40, CD80, CD86 in vitro; B) Secretion of TNF-α in macrophage (RAW 264.7) suspensions measured by ELISA; C) secretion of TL-6; and D) secretion of IL-12 in DC suspensions measured by ELISA. Data represent mean±SD (n=3, *p<0.05 vs. CpG group, #p<0.05 vs. MSN-CpG group).

FIGS. 14A-14E depict A) quantification of DC maturation markers CD40, CD80, and CD86 in the popliteal lymph node (n=3); B) tetramer staining analysis of gp100-specific T cells (n=3); C) illustration of the prophylactic and therapeutic experiment design; D) prophylactic effect of nanovaccines on survival rate (n=6); and E) effect of nanovaccines with or without the checkpoint blockade inhibitor anti-CTLA-4 on survival rate (n=6). Data represent mean±SD (*p<0.05 vs. CpG group, #p<0.05 vs. MSN-CpG group, &p<0.05 vs. bulk MSN-CpG@CM group).

FIGS. 15A-15B depict secretion of A) IL-6 in DCs isolated from popliteal lymph nodes after vaccination with nanovaccines or control formulations; and B) IL-12 in DCs isolated from popliteal lymph nodes after vaccination with nanovaccines or control formulations. Data represent mean±SD (n=3, *p<0.05 vs. CpG group, #p<0.05 vs. MSN-CpG group).

FIGS. 16A-16B depict A) average tumor sizes; and B) individual tumor growth kinetics for nanovaccines in prophylactic melanoma model (n=6).

FIGS. 17A-17B depict A) average tumor sizes and B) individual tumor growth kinetics for nanovaccines with or without the checkpoint blockade inhibitor anti-CTLA-4 in melanoma model (n=6).

### DETAILED DESCRIPTION

### Definitions

The following definitions are provided for the purpose of understanding the present subject matter and for constructing the appended patent claims.

It is noted that, as used in this specification and the appended claims, the singular forms “a”, “an”, and “the” include plural references unless the context clearly dictates otherwise.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

A method for synthesizing cell membrane-cloaked particles can include coating core particles with cell membrane materials using flash nanocomplexation (FNC). FNC is a turbulent mixing and self-assembly method that can produce cell membrane-coated nanotherapeutics in a reproducible and scalable manner. FNC can induce electrostatic interactions which rapidly homogenize the charged core particles with the negatively charged cell membrane materials to achieve a uniform coating of the core particles. The turbulent mixing generally involves continuous rapid mixing in a confined space and can be associated with a Reynolds number that is larger than 1600. The continuous mixing achieves homogeneity and consistency of the product particles. Compared to batch-mode processing methods, such as dialysis, emulsification, and slow precipitation, flash self-assembly provides better control of particle size and better reproducibility, scalability, and throughput capacity. FNC is a kinetically controlled mixing process which exploits polyelectrolyte complexation-induced phase separation. In the present method, the nanocomposites can undergo self-assembly via physical interactions such as electrostatic interactions and hydrogen bonding, and are formed within milliseconds or microseconds in flash mixers. The fluid dynamics of the flash mixers can be turbulent. In other words, the interaction of the liquid solutions can be robust within the mixer for better flow convection, allowing rapid, homogenous, and effective mixing for reactions.

The method can include loading a cell membrane material and a core particle into a confined mixing cavity and turbulent mixing of the cell membrane material and the core particle in the cavity to provide the cell membrane-cloaked particles. The turbulent mixing can achieve a turbulent intershearing flow in the confined cavity. Turbulent flow, as used herein, refers to a high dynamic flow (e.g., non-laminar flow) that renders a mixing profile with a high Reynold number. One of the characteristics of the turbulent flow is an intershearing flow or turbulent intershearing flow which facilitates efficient mixing and diffusion for achieving homogeneous mixing of materials. The method can provide coated particles having a coating thickness ranging from about 5 nm to about 20 nm. Notably, the present method can be used to coat positively charged nanoparticles. The flash-based cell membrane coating is a superior biomimetic preparation platform to standardize the membrane-coating protocol and to meet clinical translation requirements. The method can be used for synthesizing vaccines, drug and gene delivery, as well as a wide range of other applications.

The core particles can include at least one of nanoparticles and microparticles. The core particles can include, for example, silica particles, biodegradable polymer particles, DNA-polymer polyplex particles, and chemotherapeutic nanocrystals. The polymer can include, for example, poly(lactic-co-glycolic acid) (PLGA) and polyethyleneimine (PEI)-plasmid. The core particles range in size from about 50 nm to about 2 μm with surface charge of the core particles varying from about −50 mV to about +50 mV. The core particles can be loaded with an adjuvant. The cell membrane coating materials can be from any suitable cell line. For example, the cell membrane coating materials can include cell membrane fragments obtained from cancer cells, non-immune cells, and immune cells. Exemplary cell lines from which the cell membrane fragments can be obtained include, for example, CaCo-2, HepG2, MCF-7, RAW 264.7, HEK, HeLa, HITC, B16-F10, RBC, MSC. The cell membrane material can include a tumor-associated antigen.

An embodiment of the present teachings is directed to a biomimetic vaccine comprising the cell membrane-cloaked particles prepared according to the present methods. The biomimetic vaccine can including a core particle cloaked with a cell membrane material including a tumor-associated antigen. The core particle can be loaded with an adjuvant. In an embodiment, the core particle comprises a mesoprous silica nanoparticle (MSN). In an embodiment, the adjuvant is CpG.

In an embodiment, the cell membrane material and the core particles can be loaded in a confined mixing cavity. In an embodiment, the confined mixing cavity can include a multi-inlet vortex mixer (MIVM). In an embodiment the core particles can be selected from mesoporous silica nanoparticles (MSNs) and silica dioxide microparticles. The MSNs can be modified with an amine group to endow the MSNs with a positive surface charge. In an embodiment, the MIVM comprises 4 inlets. Turbulent mixing of the cell membrane material and the core particles can be performed to achieve a flow rate in each inlet ranging from about 5 mL/min to about 40 mL/min. The mass ratio of the cell membrane to core particle can range from about 0.1 to about 100 for coating optimization. The cell membrane material and the core particles can be mixed at a high Reynold number, e.g., typically larger than 1600, which constitutes highly turbulent mixing, in the confined mixing cavity at room temperature to achieve a well-controlled cell membrane cloaked particle or coated particle. The coated particle can have a coating thickness ranging from about 5 nm to about 20 nm, depending on the flow rate and mass ratio used, and the polydispersity can be lower than 0.2. The coated particles can have a relatively high colloidal stability.

FNC exploits the dynamic mixing of nanocomposites that undergo self-assembly via physical forces such as electrostatic interactions, whereby charged nanomaterials assemble to form nanoparticles (NPs) or to modify a NP or microparticle surface. FNC can be used for mixing the cell membrane fragments and synthetic backbone materials for the robust and scalable production of cell membrane-coated particles. Uniform coated nanoparticles can be fabricated using FNC by optimizing the cell membrane/NP mixing ratio, flow rate, and composition. For example, the flow rate and mass ratios of the different mixes can be varied.

A significant driving force of the present method is the electrostatic interactions induced by FNC. The electrostatic interactions rapidly homogenize the negatively charged cell membrane with the disparate charged nanoparticle core. Coating of the particle core is achieved while the cell membrane fragments self-assemble and land onto the outer surface of the particle uniformly. It should be noted that homogeneous cell membrane coating on a positively charged nanoparticle surface has not previously been reported.

The present method can standardize the otherwise unpredictable process of membrane coating of drug nanoparticles, increasing the reliability of cell-targeted drug delivery. In experiments described herein, the potency of flash-based membrane coating (FNC) method was compared with bulk sonication, a conventional method for cell membrane coating. The FNC-produced cell membrane-coated nanoparticles demonstrate lower aggregation, polydispersity, and zeta potential, than nanoparticles prepared by bulk-sonication. Mesoporous silica nanoparticles (MSN) were modified with an amine group to endow the MSNs a positive surface charge. It was demonstrated that FNC-based cell-membrane coating of positively charged MSNs has a wider coating ratio range (membrane to core) than the bulk-sonication method. As such, the present method achieves more complete and homogeneous coating than conventional bulk-sonication methods.

In an embodiment, turbulent mixing of the cell membrane fragments and the core particles can be conducted in a mixing microchamber or other confined cavity. For example, a multi-inlet vortex mixer (MIVM) can be used. The inlet jets of the MIVM can produce kinetic energy which can transport cell membrane fragments and synthetic backbone materials into regions of small turbulent eddies and intershearing layers for better flow convection and hence faster coating. The mixing ratio and flow rate can then be optimized to produce a uniform coating. The turbulent mixing can have a Reynold number larger than 1600. During turbulent mixing, the cell membrane fragments can be evenly distributed around the particle for homogeneous coating because the mixing time (τ_{mixing}), during which cell membrane fragments and core particles are mixed homogenously, can be much shorter than the interacting time (τ_{coating}) between the cell membrane fragments and the core particles. Since the cell membrane material and nanoparticles are introduced to the flow chamber by different FNC inlets, the cell membrane fragments can be distributed around the nanoparticle evenly before they electrostatically interact with the nanoparticle surface.

The method can be used for coating a variety of core particles with cell membranes. The method can be used to coat cationic particles, for example. The method can be used to prepare nanovaccines. For example, the method can produce cancer nanovaccines, such as B16-F10 cancer cell membrane-coated mesoporous silica nanoparticles (MSNs) loaded with the adjuvant CpG.

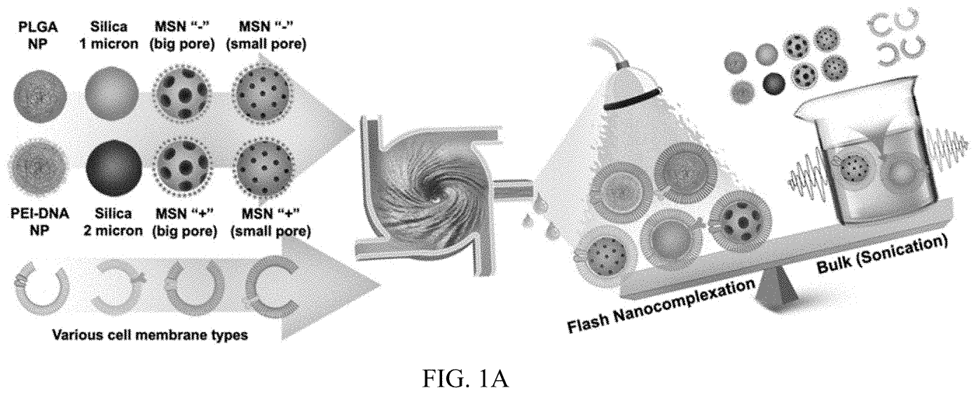

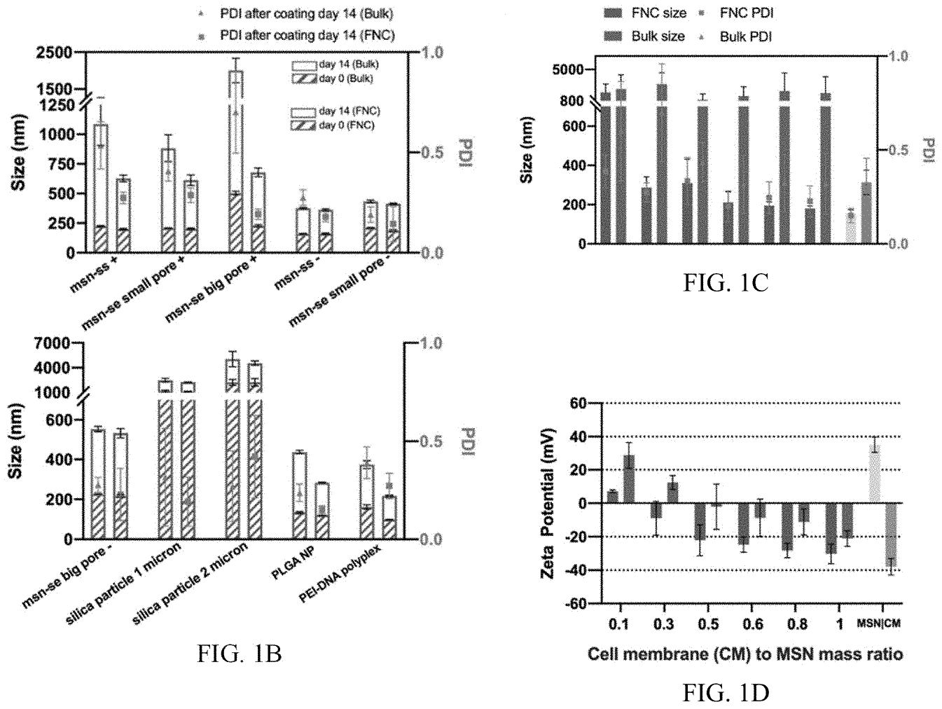

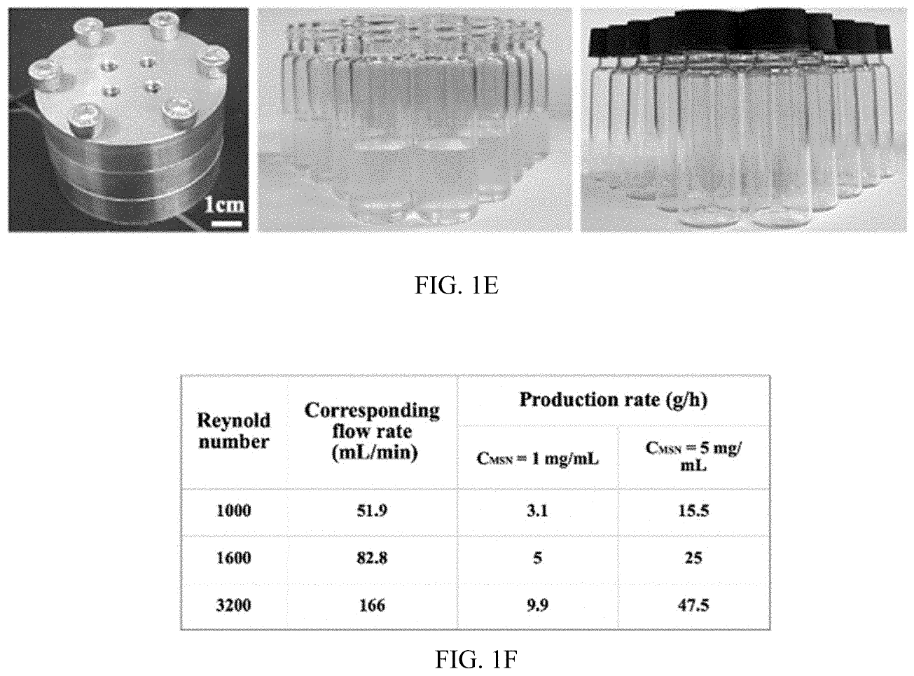

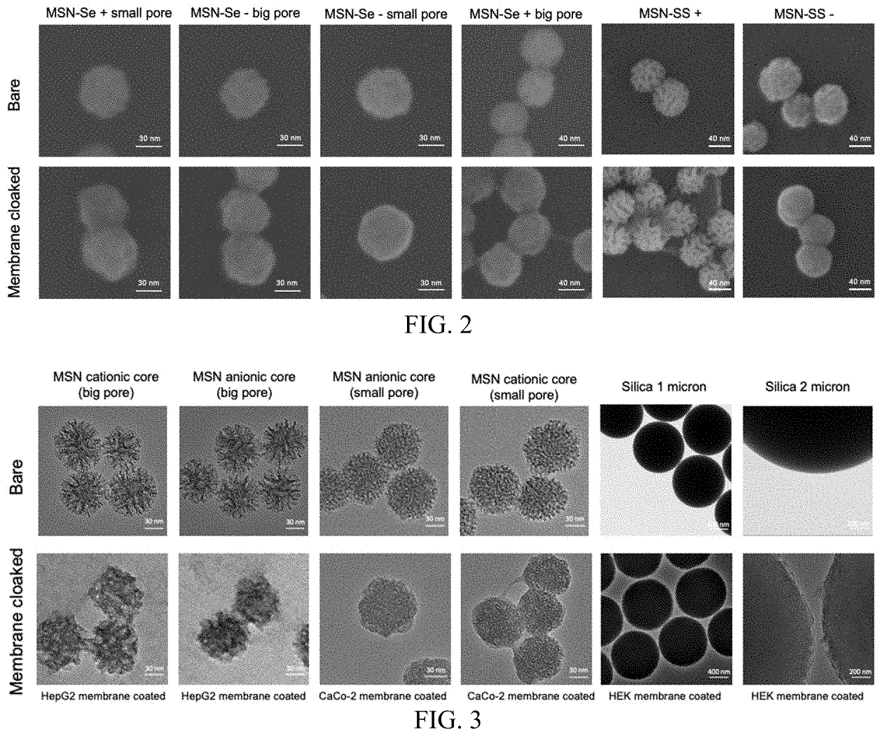

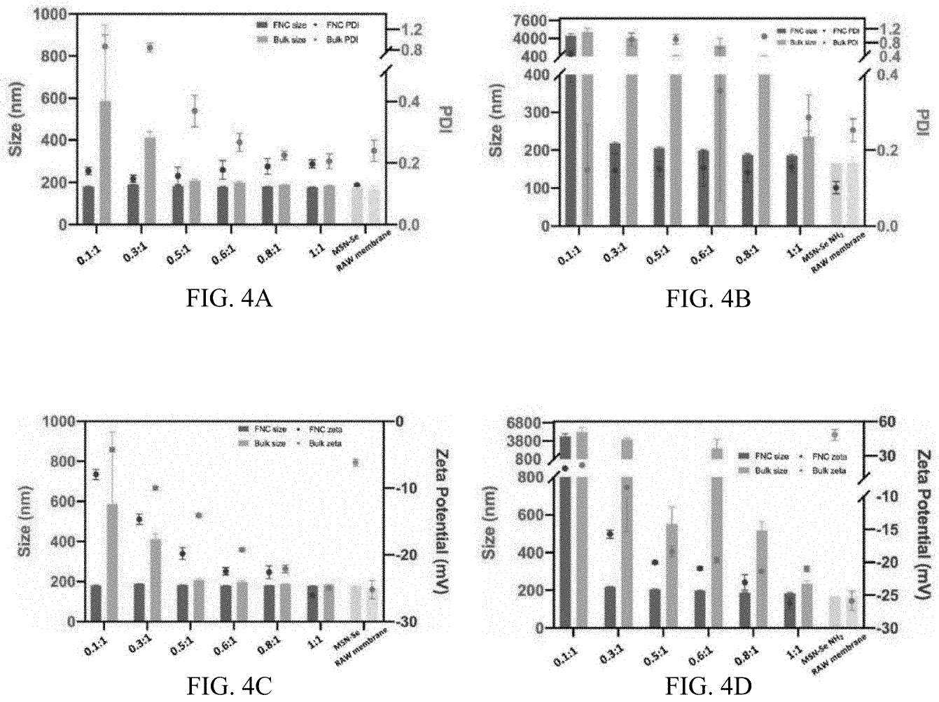

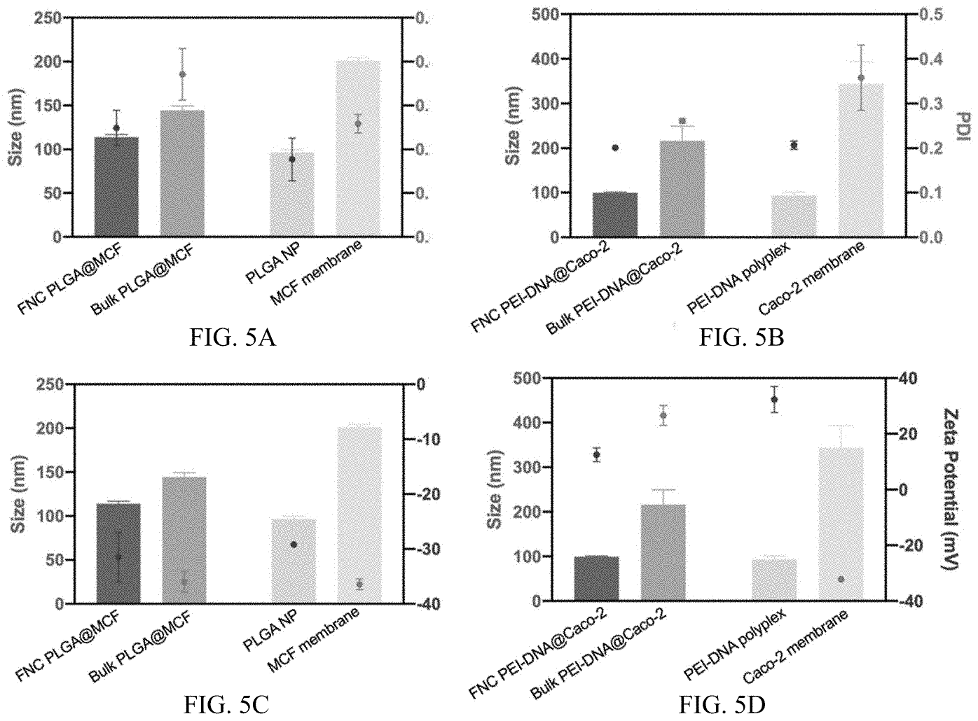

The FNC cell membrane coating process relies on electrostatic interactions between cell membrane fragments and core materials to form “right-side-out” membrane-coated products. The “right-side-out” orientation refers to an orientation in which the membrane-bound proteins are still exposed on the outside after membrane coating. With prior coating methods, it was difficult to apply cell membranes uniformly to cationic surfaces due to the collapse of the fluidic lipid bilayer and disordered structure, resulting in particle aggregation. Further, the mixing time (Tixiny), within which cell membrane fragments and backbone materials are mixed homogenously, was much larger than the interacting time (τ_{coating}). Thus, only a fraction of the cell membrane fragments were available to participate in coating the positively-charged backbones, leading to heterogeneous coating and irreversible aggregation. To test the hypothesis that FNC can improve the uniformity of coating by reducing τ_{mixing}, FNC and bulk sonication methods were compared in coating cationic MSNs. Four different operators who were new to the protocol of both methods of performing cell membrane coating of cationic MSNs performed the method. FIG. 1A shows coating outcomes of both FNC and sonication approaches for coating nanoparticles using different nanoparticle core materials and cell membrane types. Ten particulate cores with different size, pore structure, and surface charge were selected, which included poly(lactic-co-glycolic acid) (PLGA), polyethyleneimine (PEI)-plasmid, and silica particles. Cancer, non-immune, and immune cells were used to obtain the cell membrane coating materials. The core-shell structure of the resulting cell membrane-coated particles was confirmed using electron microscopy (FIGS. 2 and 3). Increases in particle size and surface charge were measured using dynamic light scattering (DLS) (FIGS. 4A-4D and 5A-5D).

FIG. 1B shows a plot of size change of various particles immediately after coating versus change at two weeks after coating and sitting in water. A remarkable difference in membrane coating homogeneity and in particle stability between the products of the two methods was demonstrated. Across a spectrum of NPs of cell membranes, FNC products showed a smaller size change and better particle colloidal stability than the bulk-sonication products. Specifically, for many of the MSN subtypes and PLGA nanoparticles, the aggregation seen at day 14 was significantly reduced. The lower polydispersity index (PDI) for FNC products immediately after coating demonstrated a more complete coating. In the sonication method, ultrasound wave-energy pulverizes the cell membrane structure, and membrane fragments re-assemble around the nanoparticle backbone. Such coating protocol is not standardized for bulk containers because the sonication power-frequency is not balanced or optimized, and the quantity of cell membrane charge over the backbone surface is not well-controlled. While electrostatic interactions are the driving force in both approaches, the FNC method achieves ultra-fast and homogeneous coating by using turbulent mixing, including turbulent intershearing flow in the microchamber. This dynamic mixing is more effective than sonication in breaking cell membranes into small fragments and mixing the components to achieve even coating.

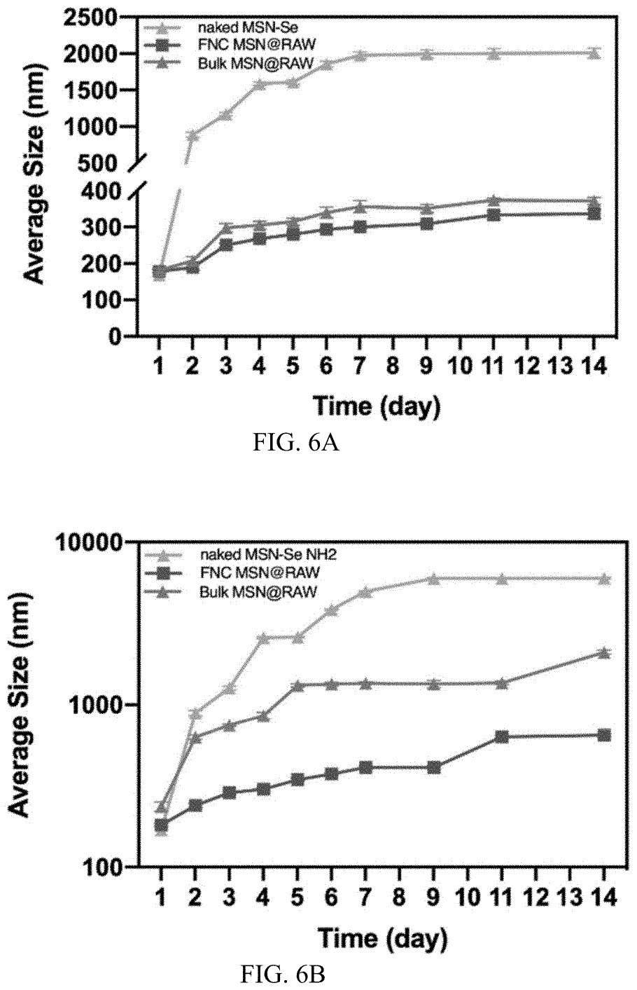

FNC products showed excellent dispersion (low PDI) at nearly all membrane/MSN ratios (FIG. 1C), while sonication products showed significantly higher PDI values. FNC also yielded better nanoparticle charge conversion than sonication, which suggests a more complete cell-membrane coating (FIG. 1D). The surface charge of a completely coated nanoparticle resembles the intrinsic charge of cell-membrane vesicles, whereas incomplete coating partially reveals the charge of nanoparticles and neutralizes the zeta potential. The membrane-coated MSNs<200 nm diameter also showed better colloidal stability in serum-containing solutions when produced with FNC than with sonication (FIGS. 6A-B). These results indicate for the first time that even cationic nanoparticles can be effectively coated with cell membranes.

As efficient manufacturing is a key factor in clinical translation of biomimetic therapeutics, the scale-up capability of the FNC procedure was investigated by testing the rate of production of cell membrane-coated MSNs. Using a four-inlet MIVM with the total flow rate of 120 mL/min (30 mL/min for each stream), 40 mL of 20 mg (total weight) membrane-coated MSNs were prepared in just 20 seconds (FIG. 1E). At the same MSN concentration, with a total flow rate of 166 mL/min, about 120 g of membrane-coated NPs were produced in a single day (FIG. 1F). It should be noted that the rate of production in a laboratory setting is typically 5-50 mg per batch and <5 g per day when using bulk mixing and sonication.

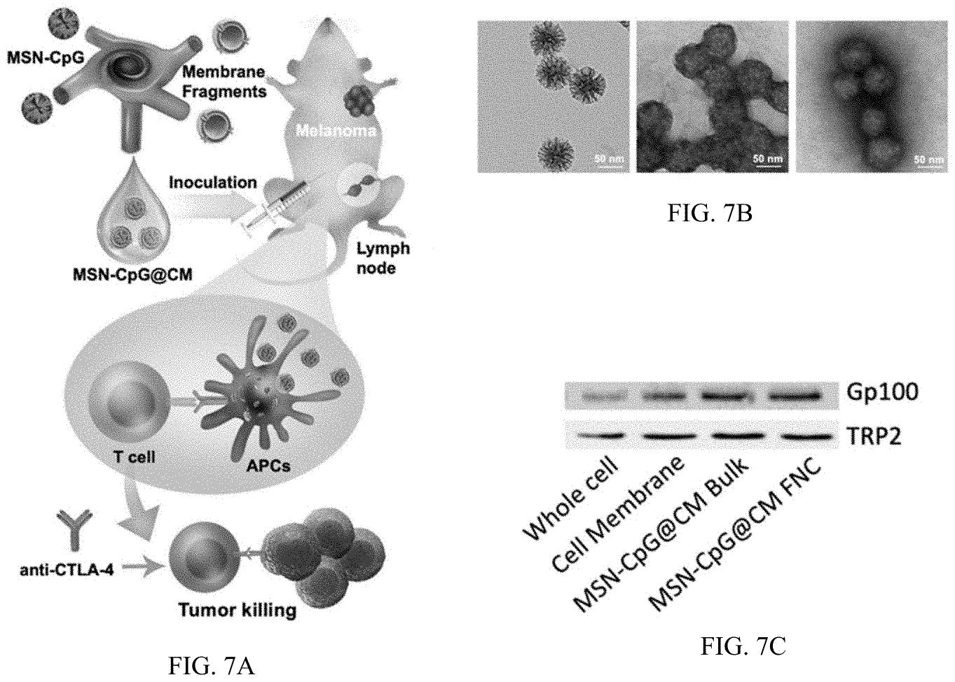

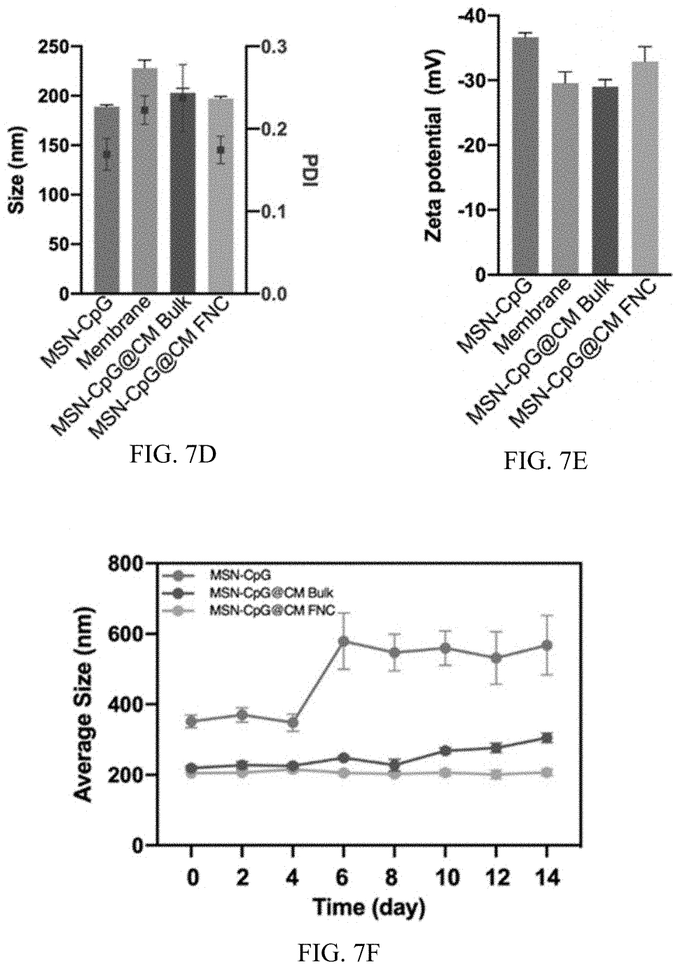

Cell membrane-coated nanoformulations show promise for use in cancer immunotherapy. Cancer vaccines can be created by combining tumor-associated antigens and immune-activating adjuvants. The presentation of tumor-associated antigens on cancer cell membrane-coated backbone materials together with delivery of adjuvants such as CpG can generate tumor-specific immune responses and lymph node targeting. The present inventors previously fabricated multiple stimuli-responsive and biodegradable diselenide-bridged MSNs for efficient delivery of biomacromolecules for cancer therapy. Using the methods described herein, a biomimetic vaccine (MSN-CpG@CM) was synthesized by coating large-pore MSNs loaded with the adjuvant CpG with cancer cell membrane fragments containing tumor-specific antigens (FIG. 7A). CpG 1826 was encapsulated in amine-modified MSNs (MSN-NH₂) for maximum loading. B16-F10 mouse melanoma cell membranes were selected for the coating. The FNC and bulk mixing/sonication approaches were systematically compared to determine whether they were capable of scalable production of these biomimetic cancer vaccines, and their therapeutic efficacy was evaluated in vitro and in vivo.

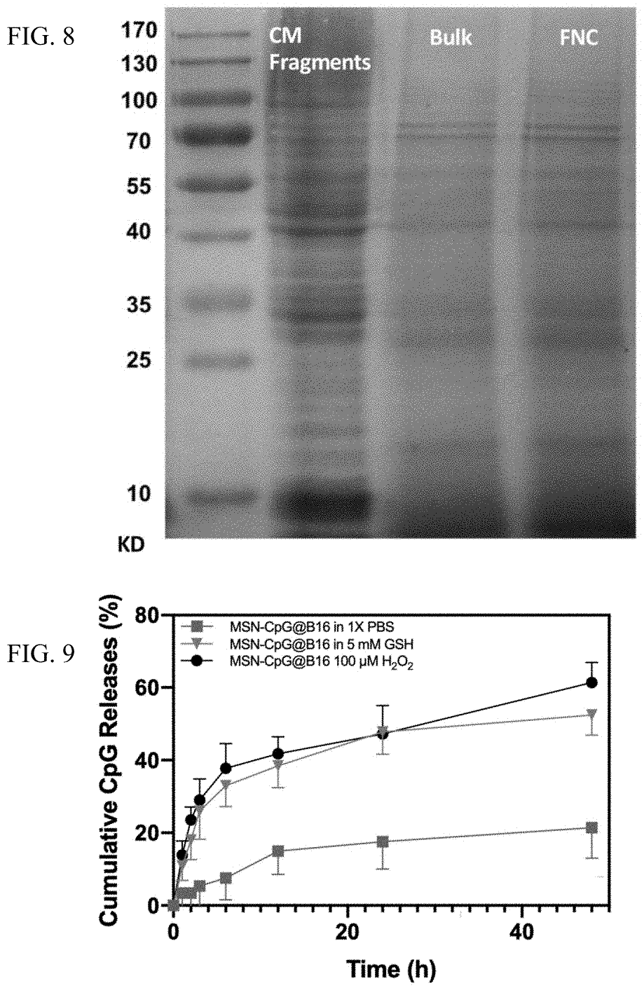

CpG-loaded MSNs were coated with B16-F10 cell membrane fragments using the FNC platform with a turbulent MIVM micromixer and using bulk sonication. A membrane-to-NP mass ratio of 2:1 was selected since this value is often reported as the optimal ratio for cell membrane coating. The surface morphologies of the CpG-loaded MSNs before coating (MSN-CpG) and after coating (MSN-CpG@CM) using the two methods are shown in TEM images (FIG. 7B). The tumor-associated antigen gp100 is specific for targeting melanoma in drug and vaccines. The presence of gp100 in the membrane coating of the MSN-CpG@CM particles was confirmed by Western blot (FIG. 7C). Other B16-F10 cell membrane proteins were also found in the coating of the MSN-CpG@CM particles (FIG. 8). An increase in nanoparticle size and an inverted zeta potential after coating also indicated the presence of a cell membrane coating (FIGS. 7D and 7E). Smaller PDI values were observed for the MSN-CpG@CM particles when using FNC than when using bulk sonication. For both methods, significant aggregation was observed for naked NPs over the two-week stability evaluation period, whereas the membrane-coated NPs maintained consistent size (FIG. 7F). The improved colloidal stability might be explained by the symmetrical charge repulsion between cell membrane-coated NPs. In addition, a high CpG cargo-loading capacity and GSH/reactive oxygen species (ROS) dual-responsive CpG release were observed for the MSN-CpG@CM (FIG. 9), indicating their potential for use in stimuli-responsive immunotherapeutic delivery.

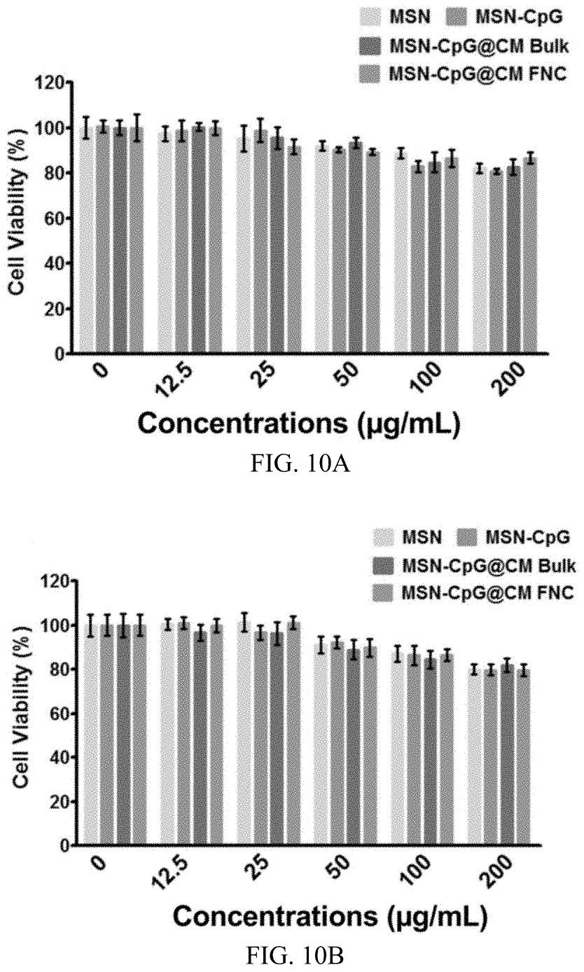

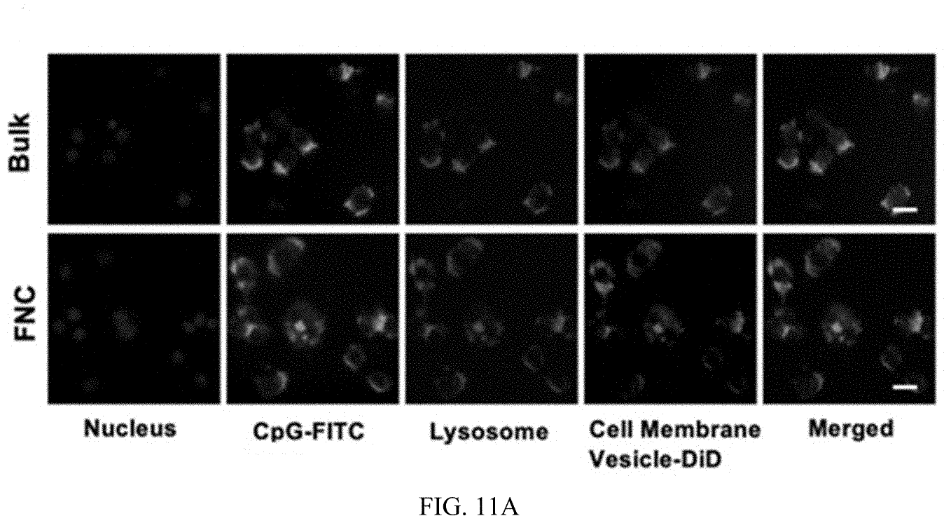

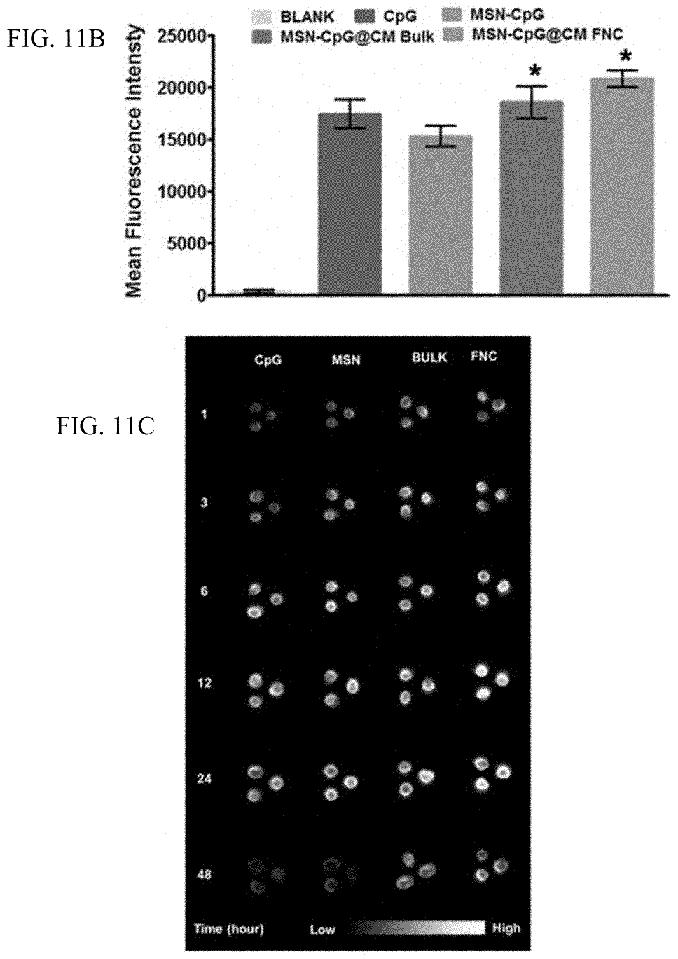

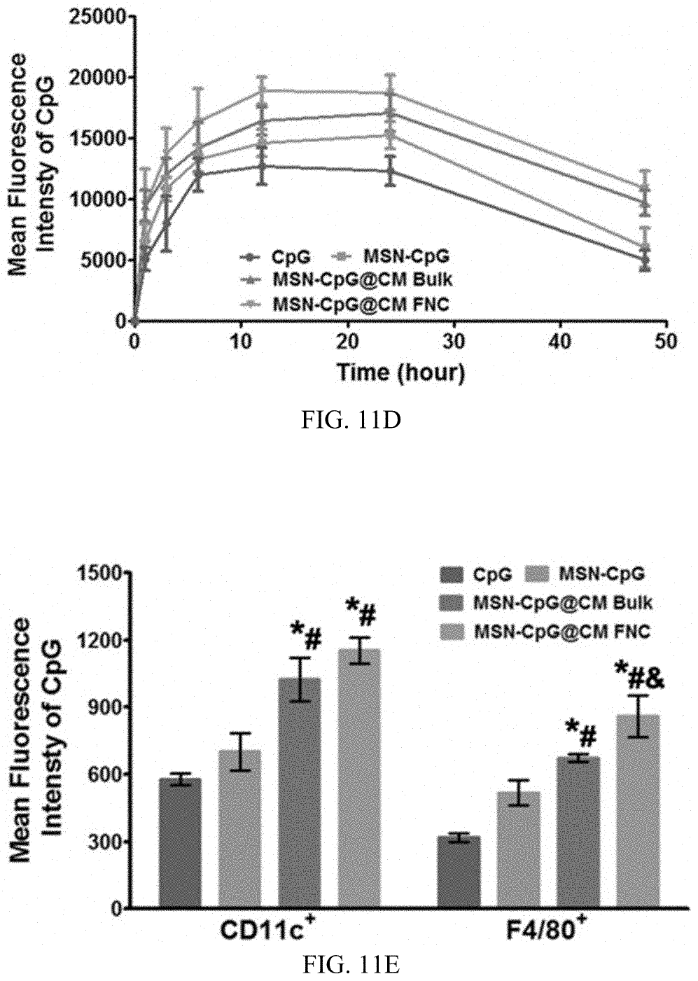

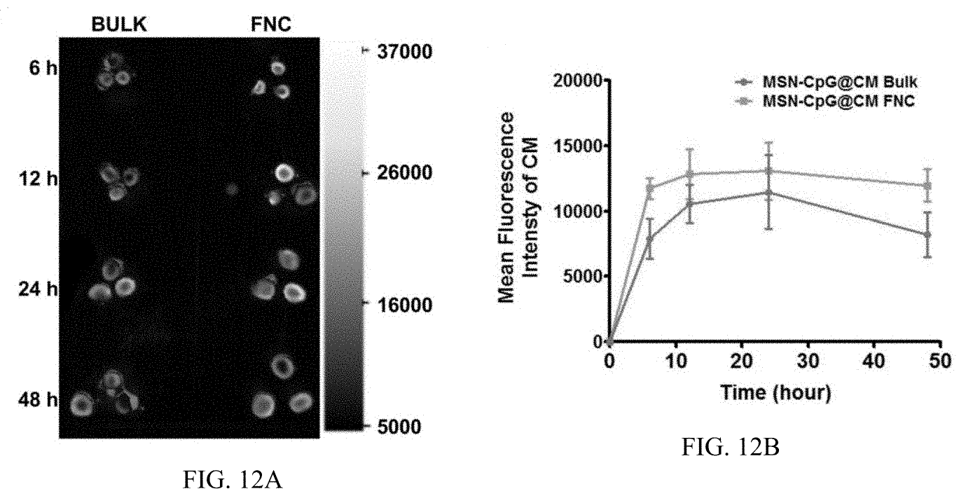

It was confirmed that MSN-CpG@CM at <50 μg/mL showed no significant cytotoxicity using two types of antigen-presenting cells (APCs) (FIGS. 10A-10B). A high degree of intracellular colocalization of CpG-loaded MSNs and cancer cell membrane proteins were observed in endosomes/lysosomes after 3 h of uptake (FIGS. 11A-11E), further verifying the structural integrity and stability of the MSN-CpG@CM. The uptake of MSN-CpG@CM by bone marrow-derived dendritic cells (BMDCs) was then investigated (FIG. 11B). Both MSN-CpG@CM groups (prepared using either FNC or bulk sonication) showed improved CpG uptake by DC cells versus naked MSN-CpG, demonstrating the APC-targeting effect in vitro. MSN-CpG@CM were then injected into mice via the foot pad, and nanovaccines were observed in the popliteal lymph node after 1 h of administration. The fluorescence signal from dye-labelled CpG peaked at 12 h after injection, and started to decrease at 24 h (FIG. 11C). Quantification of mean fluorescence intensity of free CpG, naked MSN-CpG, and MSN-CpG@CM in the lymph node confirmed this observation (FIG. 11D). Greater lymph node accumulation of MSN-CpG@CM was observed for vaccines prepared using the FNC method than vaccines produced by sonication, and further confirmed using dye-labelled membranes (FIGS. 12A-12B). In terms of targeting APC internalization, both DCs and macrophages preferred endocytosing MSN-CpG@CM to MSN-CpG, indicating specific recognition of tumor antigens by the APCs. Greater CpG accumulation in DCs and macrophages from the popliteal lymph node was observed when using FNC-produced vaccines than when using sonication-produced vaccines (FIG. 11E). Collectively, these results indicated that FNC produced a cell membrane-coated cancer vaccine with better lymph node targeting and APC accumulation than sonication.

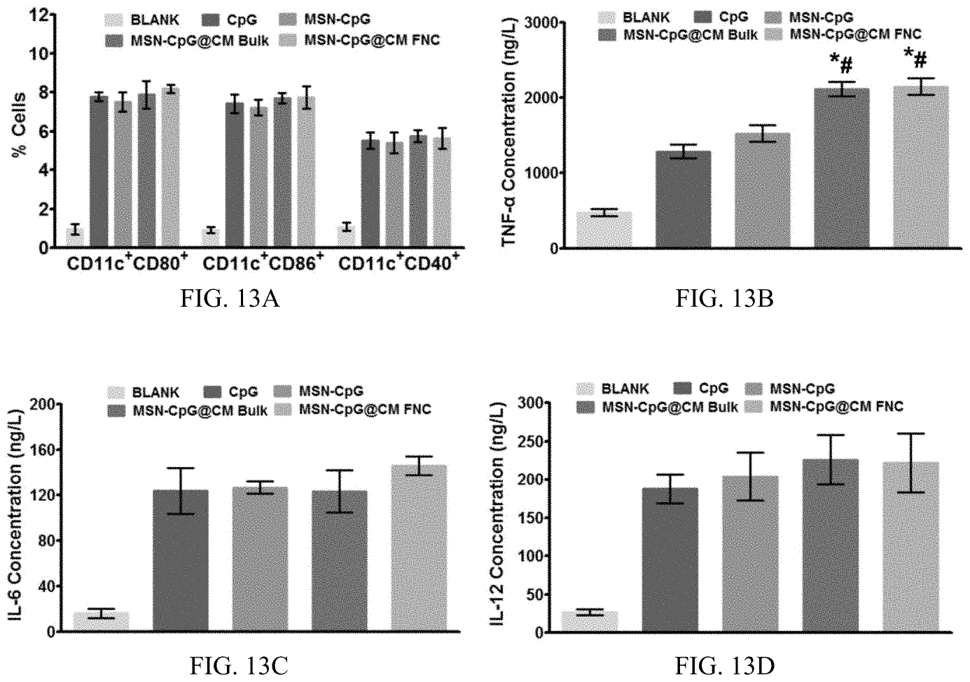

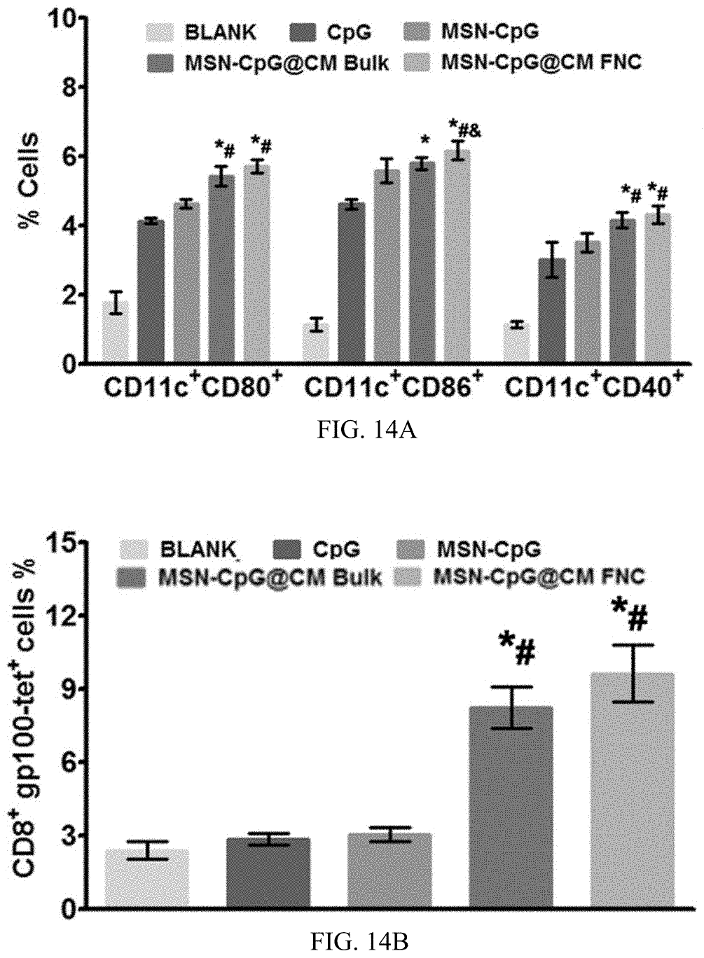

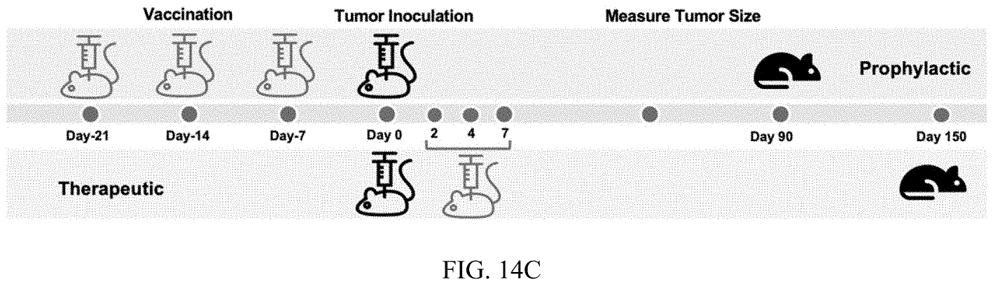

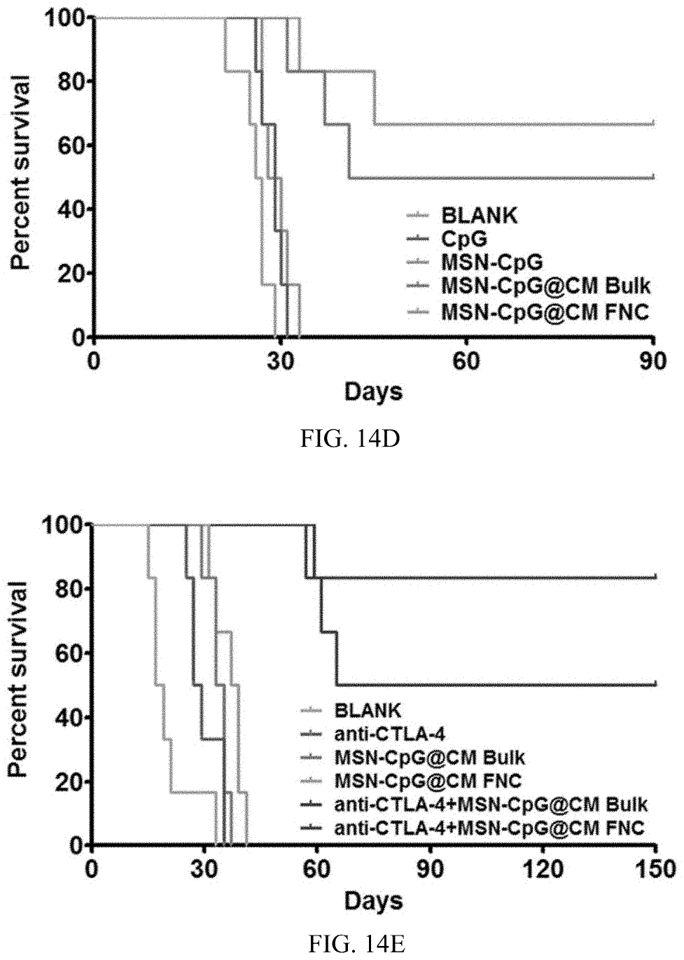

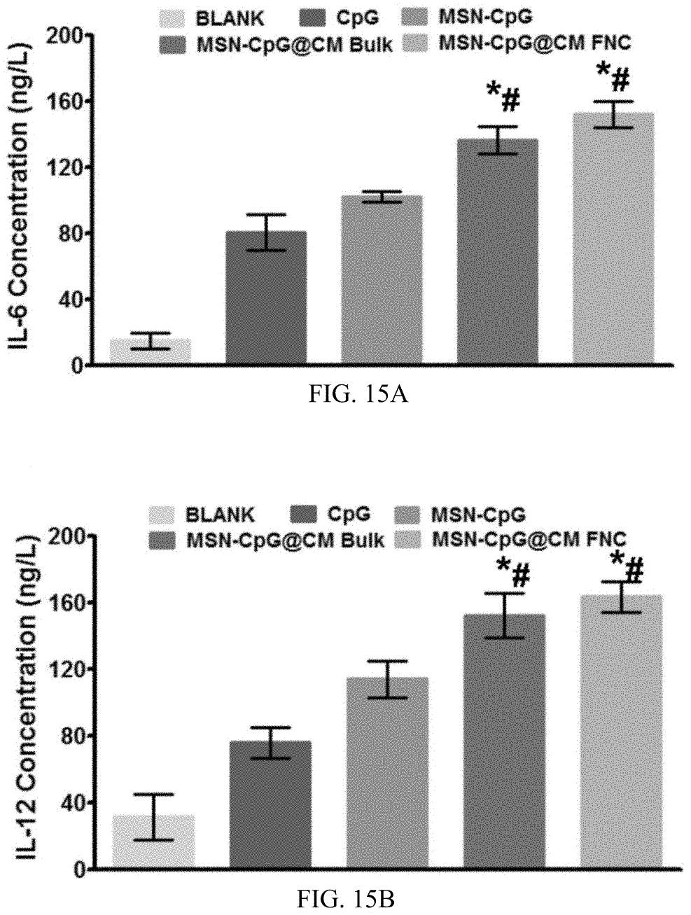

The immunostimulatory effect of MSN-CpG@CM was characterized by assessing DC maturation and the generation of antigen-specific T cells. DC maturation was assessed by measuring the expression of the costimulatory markers CD80, CD40, and CD86. The secretion of TNF-α, IL-6, and IL-12 from APCs were also determined in vitro (FIGS. 13A-13D). In lymph node, CpG alone and MSN-CpG induced less potent DC maturation than MSN-CpG@CM (FIG. 14A). MSN-CpG@CM produced using FNC induced greater DC maturation and secretion of IL-6 and IL-12 than MSN-CpG@CM produced using sonication (FIG. 14A, and FIGS. 15A-15B). Importantly, FNC-formulated MSN-CpG@CM promoted greater generation of T cells specific for gp100 than sonication-formulated MSN-CpG@CM (FIG. 14B), indicating better presentation of gp100 antigen for T-cell activation. Together, these results indicated that the FNC-produced cancer vaccine could stimulate DC antigen presentation and a tumor antigen-specific T cell response.

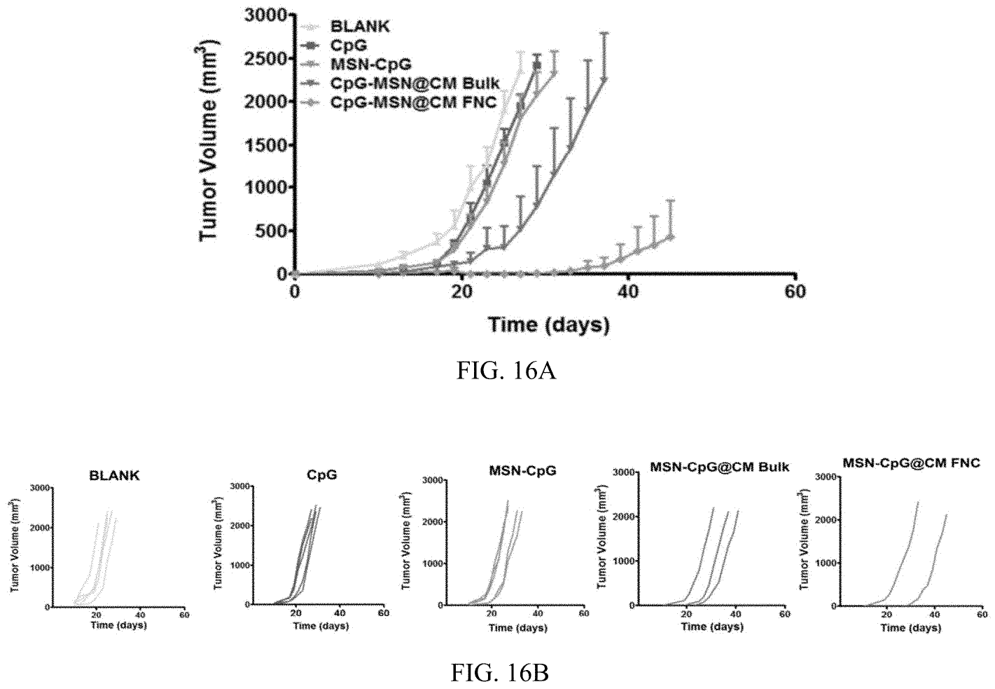

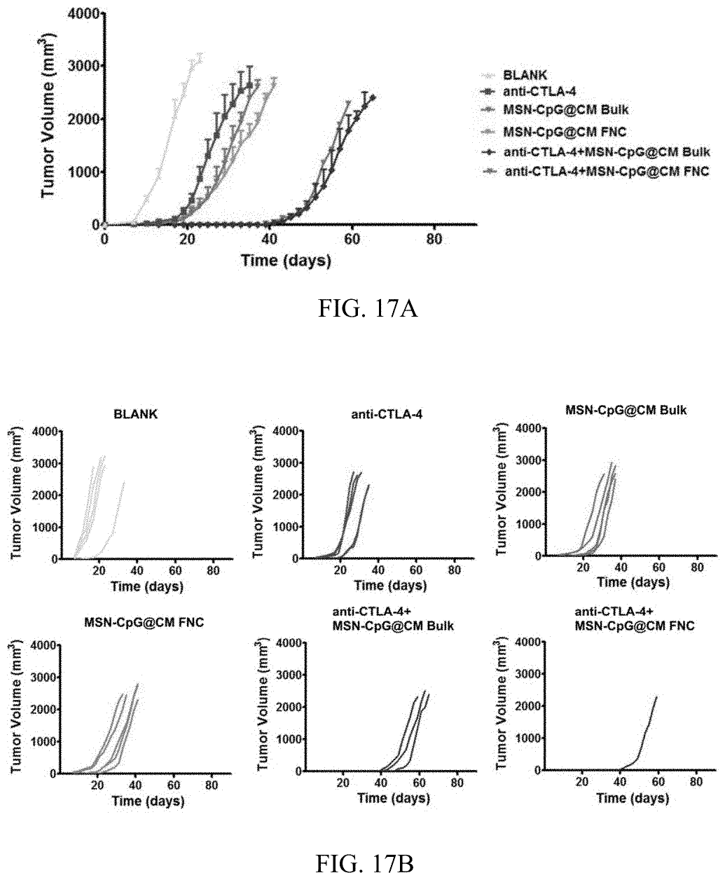

APCs responses, specific immune activation, and prophylactic tumor growth inhibition in vivo were evaluated using a 16-F10 murine model (FIGS. 14C-14D). Mice were vaccinated using different nanoformulations and tumor growth was monitored for up to 40 days. MSN-CpG and free CpG had no significant protective benefit, consistent with previous studies; both treatments showed a median survival of 29 d, similar to the median 26.5 d survival for the negative control. Both FNC- and sonication-produced MSN-CpG@CM groups showed tumor growth inhibition, but the FNC-produced vaccine had a much greater inhibitory effect and longer survival (FIGS. 16A-16B). Performance of the MSN-CpG@CM was assessd with and without the immune checkpoint-blocking antibody anti-CTLA-4. Without anti-CTLA-4, the median survival was extended from 18 d for the blank control group to 34 d for the bulk sonication MSN-CpG@CM group and 38 d for the FNC MSN-CpG@CM group (FIGS. 14C and 14E). With anti-CTLA-4, the median survival was over 150 days for both FNC and sonication MSN-CpG@CM groups, indicating that combined immunotherapy produced synergistic antitumor effects. The combined therapy using FNC-produced nanovaccines with anti-CTLA-4 had the strongest antitumor effect (FIGS. 17A-17B).

The present methods provide a nanoformulation platform for fabricating diverse cell membrane-based biomimetic NPs in a facile, reproducible, and scalable manner. The FNC platform leverages dynamic turbulent mixing to homogeneously blend and uniformly distribute cell membrane fragments around NP surfaces. FNC can be used to coat both negatively- and positively-charged particles with cell membranes. By reducing batch-to-batch variation and production time, the FNC method may enable standardization of the cell-membrane coating process for clinical translation. FNC-produced MSNs loaded with CpG adjuvant and coated with a cancer cell membrane exhibited enhanced accumulation in lymph nodes and immune activation, and greater tumor growth inhibition alone and in combination treatment with the immune checkpoint-blocking antibody anti-CTLA-4 in an in vivo melanoma model. High-throughput manufacturing of nanomedicine can pose a challenge for clinical and industrial translation. The cell membrane-coating method described herein addresses this challenge. The advantages of FNC include (1) automation, using an easy-to-transfer protocol; (2) reproducibility, reducing batch-to-batch variation; (3) user-friendliness, obviating training requirement, and (4) scalable manufacturing, facilitating clinical and industrial translation.

The present teachings are illustrated by the following examples.

### EXAMPLES

### Materials and Methods

Tetraethyl orthosilicate (TEOS), bis[3-(triethoxysilyl)propyl]tetrasulfide (BTESPT), 7-chloropropyl trimethoxysilane (CP), 3-aminopropyltriethoxysilane (APTES), etyltrimethylammonium tosylate (CTAT), triethanolamine (TEAH3), triethanolamine (TEA), carboxyl-terminated 50:50 poly(lactic-co-glycolic) acid, fluorescein isothiocyanate (FITC), polyethylenimine linear (Mn 2500), succinic anhydride, carbodiimide hydrochloride (EDC), sulfo-N-hydroxy succunimide (sulfo-NHS), and silica dioxide microparticles that were 1 and 2 microns in size were purchased from Sigma-Aldrich Co. (St Louis, Mo., USA). Hoechst 33343 bisbenzimide H-33343 trihydrochloride was purchased from VWR. LysoTracker Red DND-99 and Vybrant DiD Cell-Labeling Solution (V22887) were purchased from Thermo Fisher Scientific. ODN 1826-TLR9 ligand and ODN 1926 FITC were purchased from InvivoGen. Anionic and cationic MSN-Si (Si—Si) with an average NP diameter of 80-100 nm; anionic and cation MSN-Se (Se—Se) with an average NP diameter of 80-100 nm; SiO₂ microparticles with sizes of 1 m and 2 m; GFP plasmid-PEI NPs and PLGA NPs were selected for membrane coating. All types of MSN (e.g., Si—Si and Se—Se, disulfide and diselenide bridged MSN, respectively) with different pore sizes were synthesized in the lab using previously described methods. Optimal CpG loading was achieved with the MSN-to-CpG mass ratio of 5 to 1. A greater than 96% encapsulation efficiency of CpG in MSN-NH₂ was obtained. CpG released from MSNs were evaluated in PBS or 5×10⁻³ M GSH or 1×10⁻⁴ M H₂O₂ from 0 to 48 h. Briefly, CpG-MSN solution was placed on a shaker with 200 rpm. At each timepoint, the solution was centrifuged and the supernatant was analyzed by UV-Vis. PLGA NPs were prepared by flash nanoprecipitation method as previously reported using a two-inlet confine impingement jet mixer (CIJ). GFP-PEI nano-polyplexes were fabricated as previously described. CIJ mixer was designed according to literature and fabricated in Columbia University Biomedical Engineering machine shop. A four-stream multi-inlet vortex mixer (MIVM) was manufactured according to the literature. FNC-based cell membrane coating was achieved in the manufactured four-stream MIVM. Specifically, B16-F10 cell membrane to MSNs ratio of 0.5 and 1 were prepared. Cell membrane and CpG-MSN were introduced into the MIVM respectively. A total of 120 mL/min flow rate was applied to prepare membrane-coated nanoparticles. Infusion/withdrawal PHD ULTRA 4400 pumps were obtained from Harvard Apparatus.

### Example 1

### Cell Membrane Derivation

All cell lines were maintained in DMEM supplemented with 10% FBS and 1% antibiotic/antimycotic solution and incubated at 37° C. with 5% CO₂ in T175 tissue culture flasks. Cells were trypsinized, washed and suspended within PBS. To obtain cell membrane-derived fragments, a sequential centrifugation method was applied after lysis and homogenizing of the cells. The resulting membrane pellets were washed and suspended. The total membrane protein contents derived from different cell lines were quantified using BCA protein assay kits separately. Derived cell membrane vesicles were stored in DI water or PBS solution at −80° C. until further use.

### Example 2

### Cell Membrane Coating and Nanoformulation Characterization

All particles and cell membrane fragments were prepared and well dispersed in independent solutions. The cell membrane vesicles received adequate sonication treatment. Particle solutions and cell membrane fragments were introduced into the different inlets of the multi-inlet vortex mixer (MIVM).

CaCo-2, HepG2, HEK 293 cell-membranes were exploited to coat MSNs (small pore), MSNs (big pore) and silica dioxide microparticles respectively, with the membrane/particle mass ratio of 1. Also, CaCo-2 membrane was used to coat PEI-DNA nanocomplexes with the membrane/particle mass ratio of 1. For the subsequent cationic MSN 4 coating experiments, both RAW264.7 and B16-F10 membrane were applied with different membrane/MSN mass ratios. Four different operators without sufficient training on cell-membrane coating performed the MSNs coating experiment comparing the FNC to the bulk sonication method. The flow rate in each inlet was maintained within a range of 5-40 mL/min. The mass ratio of the cell membrane to nanoparticle varied from 0.1 to 100 for coating optimization. Coating was achieved by mixing the membrane and “nano-core” under high fluidic dynamic profile in the confined mixing cavity at room temperature. The typical coating thickness ranged from 5 to 20 nm, depending on the flow rate and mass ratio used, and the polydispersity was basically lower than 0.2, suggesting relatively high colloidal stability. The resulting micelle was stable in the serum-containing environment, such as complete cell culture medium.

B16-F10 membrane was used for all in vitro and in vivo studies with the initial final-product concentration of 0.5 mg/mL. The efflux was collected and allowed to settle before further coating characterization. For NPs coated using the bulk sonication method, equal volumes of cell membrane vesicles and core particles were mixed, pipetted, and sonicated in 15 mL Falcon tubes in a Branson Ultrasonic Bath sonicator at 42 kHz and 100 W for 2 min. The surface zeta potential of naked and membrane-coated particles was examined by DLS using a Malvern Zetasizer. For PLGA NPs, DLS was used to compare the size difference of bare PLGA NPs that were fabricated using double-emulsion method to the ones prepared using FNP. The size and zeta potential of MCF membrane-coated PLGA NPs using bulk-sonication and FNC were also evaluated by DLS. To test the stability of naked and membrane-coated MSNs, particles were stored for two weeks and measured by DLS every other day. Specifically, MSN-CpG@CM NPs were tested in 10% serum-containing media for the two-week stability assessment. The NPs solution concentration was 100 g/mL. For TEM characterization, samples were prepared and dried onto a carbon-coated copper grid. Membrane-coated PLGA NPs were stained with uranyl acetate before TEM imaging. Identification of gp100 tumor antigen was performed by Western blotting.

### Example 3

### Cell Culture and Cytotoxicity Assay

B16-F10 mouse melanoma cells (CRL-6457; American Type Culture Collection), RAW 264.7 mouse macrophage cells (TIB-711; American Type Culture Collection), HepG2 human liver cancer cells (HB-8065; American Type Culture Collection), Caco-2 human epithelial colorectal cancer cells (HTB-37; American Type Culture Collection), HCT-116 human colon cancer cells (CCL-247; American Type Culture Collection), and HEK 293 human embryonic 5 kidney cells (CRL-1573) were cultured for cell membrane derivation. Cells were cultured in DMEM media with 10% fetal bovine serum (Gibco) and 100 U penicillin-streptomycin.

The generation of BMDCs followed a previously published protocol. Healthy mice were euthanized using carbon dioxide asphyxiation followed by cervical dislocation. Both femurs were dissected, cleaned in 75% ethanol, and cut on both ends. Bone marrow was then flushed out of the bone with a 1 mL sterile syringe using warm DMEM media including 10% fetal bovine serum (Gibco) and 100 U penicillin-streptomycin. Cells were then pelleted at 700×g for 5 min, resuspended in BMDC growth media, including the basal media further supplemented with 20 ng/mL granulocyte/macrophage-colony stimulating factor (GM-CSF; Protech), to a concentration of 1×10⁶ cells/mL, and plated into petri plates at 2×10⁶ cells per plate. Media were half-changed every two days.

The cytotoxicity of MSN, MSN-CpG and MSN-CpG@CMs in the RAW264.7 or BMDC were assessed using an MTT assay. The assay was performed in a 96-well plate containing 5×10³ cells per well. The cells were cultured in complete medium including different concentrations (0, 12.5, 25, 50, 100 and 200 μg/mL) of substances for 24 h. The medium was replaced with fresh medium including 2.5 μg/mL MTT reagent, and an MTT assay was performed after 4 h. Then, 150 L of DMSO was added to dissolve the formazan crystals. The optical density of each well was measured by a multifunctional microplate reader at a wavelength of 490 nm. The relative survival rate (mean (%)±SD, n=6) of the cells was calculated using the following equation: survival rate (%)=(A490 treated sample/A490 untreated sample)×100%.

### Example 4

### In Vitro Uptake and Activity

For the cellular uptake study, BMDCs were collected on day 5 and plated into 24-well suspension plates. FAM-labeled CpG, MSN-CpG and MSN-CpG@CMs were added at an equivalent CpG concentration of 5 μg/mL. After 3 h incubation, the cells were washed and stained with DAPI and LysoTracker Red. 15 min later, cells were imaged by using a laser scanning confocal microscopy (CLSM). For flow cytometry, cells were collected, washed twice in PBS, and resuspended in 200 μL of 10% PBS. The cell suspension was analyzed using BD Accuri C6 plus flow cytometer. Collected data were analyzed by FlowJo software. The activity of the delivered CpG was examined using a BMDC maturation assay and cytokine release assay. BMDCs were collected on day 5, and 3×10⁶ BMDCs were plated into 6-well suspension plates in BMDC growth media. Cells were pulsed with materials for 12 h at 5 μg/mL CpG, then washed twice with fresh media. After an additional 48 h of culture, cell supernatants were collected and cytokine content was analyzed using IL-6 and IL-12 ELISA kits. The cells were then collected and washed twice. Cells were stained with FITC-conjugated anti-mouse CD11c and APC-conjugated anti-mouse CD40, CD80 or CD86. Appropriate dye-labeled antibody isotypes (Biolegend) were used for gating purposes with cells from an untreated lymph node. Data were collected using a BD FACSCelesta flow cytometer and analyzed using FlowJo software. RAW264.7 cells were plated into 6-well suspension plates at 5×10⁵ cells/well and pulsed with materials for 24 h at 5 μg/mL CpG, then cell supernatants were collected and cytokine content was analyzed using TNF-α ELISA kits.

### Example 5

### In Vitro and In Vivo NIR-II Fluorescence Imaging

All animals received care in compliance with the guidelines outlined in the Guide for the Care and Use of Laboratory Animals, and the procedures were approved by the South China University of Technology Animal Care and Use Committee. Female C57BL/6J mice were obtained at 6-10 weeks old from Hunan SJA Laboratory Animal Co., LTD.

Materials containing Cy5.5-labeled CpG or DiD-labeled membrane fragments were used to trace the distribution of nanovaccines in lymph nodes. After injecting different materials at foot pad for 1, 3, 6, 12, 24, and 48 h, female C57BL/6J mice were euthanized and their popliteal lymph nodes were collected. Dye-labeled nanovaccines, 20 μL at 1 mg/mL, were injected into both hocks of female C57BL/6J mice. At time points of 6, 12, 24, and 48 h, the popliteal lymph nodes were collected. All the lymph nodes were analyzed and quantified by In-Vivo Xtreme Imaging System (Bruker).

To further assess the cellular uptake in lymph node, dye-labeled nanovaccines were injected subcutaneously into each hock of female C57BL/6J mice. After 24 h, the popliteal lymph nodes were collected, dissociated manually by pipetting, then stained with antibodies for dendritic cells (CD11c monoclonal antibody, 12-0114-82; eBioscience), macrophages (F4/80 monoclonal antibody, 11-4801-82; eBioscience) for 30 min. Data were collected using BD FACSCelesta flow cytometer and analyzed using FlowJo software.

Dendritic cell activation following immunization with CpG, MSN-CpG, and MSN-CpG@CMs was determined by testing DC maturation and lymph node cytokine secretion. To examine DC maturation in vivo, 20 μL of each material was injected into the hock. After 24 h, the popliteal lymph nodes of all treated mice were collected into 500 μL dissociation buffer and manually dissociated. Cells were stained using PE antimouse CD11c with either APC-conjugated antimouse CD40 (124611; Biolegend), CD80 (104713; Biolegend), or CD86 (105011; Biolegend). Data were collected using a Becton Dickinson FACSCanto-II FLow cytometer and analyzed using FlowJo software. To analyze cytokine production, lymph node-derived single cell suspensions were plated with 500 μL of BMDC growth media in 24-well tissue culture plates. After 48 h, supernatant was collected and analyzed for cytokine content using TL-6 and IL-12 ELISA kits.

To assess the native generation of antigen-specific T cells, C57BL/6J mice were vaccinated subcutaneously with 20 μL of the different materials in each hock on days 0, 2, and 4. On day 10, spleens were collected and processed into single cell suspensions. After red blood cells lysis, 5×10⁶ splenocytes were plated into 6-well suspension plates and pulsed with 1 μg/mL of mouse gp100 peptide with sequence EGSRNQDWL in BMDC growth media. After 7 days, cells were collected, washed in PBS, and stained with APC-conjugated anti-mouse 8 CD8a and phycoerythrin (PE)-labeled H-2db gp100 tetramer. Data were collected using a BD FACSCelesta flow cytometer and analyzed using FlowJo software.

### Example 6

### In Vitro NIR-II Fluorescence Imaging of AIE Dots

To study the tumor prevention effect conferred by vaccination, C57BL/6J mice were vaccinated with 100 μL of the different materials at 0.1 mg/mL of CpG or equivalent, on days −21, −14, and −7. On day −1, the right flank of each mouse was shaved and, on day 0, mice (n=6) were challenged with 2×10⁴ B16-F10 cells subcutaneously on the right flank. Tumors were measured every other day and the experimental endpoint was defined as either death or tumor size greater than 2000 mm².

To study the therapeutic effect, C57BL/6J mice were first challenged on the right flank with 1×10⁵ B16-F10 cells on day 0. On day 2, 4, and 7, mice (n=6) were vaccinated subcutaneously in the same flank with 100 μL of the materials. The checkpoint blockade cocktail, consisting of 100 μg anti-CTLA4 (BP0164; BioXCell) was administered intraperitoneally on the same days. Tumors were measured every other day and the experimental endpoint was defined as either death or tumor size greater than 2000 mm².

### Example 7

### Statistical Analysis

Data were expressed as mean±SD. Differences between groups were analyzed using Student's t-test when comparing only two groups. Differences among more than two groups were analyzed using one-way analysis of variance, and the Bonferroni post hoc test was used to analyze the differences between any two groups. P<0.05 was considered representative of a statistically significant difference.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

We claim:

1. A method of using flash nanocomplexation to prepare a cell membrane-cloaked particle, comprising:
loading a cell membrane material and a core particle in a confined mixing cavity; and
turbulent mixing of the cell membrane material and the core particle in the mixing cavity to homogenously coat the core particle with the cell membrane material and provide the cell membrane-cloaked particles, the turbulent mixing achieving a turbulent intershearing flow in the confined cavity and having a Reynold number larger than 1600.

2. The method of claim 1, wherein the core particle comprises a nanoparticle.

3. The method of claim 1, wherein the core particle comprises a microparticle.

4. The method of claim 1, wherein the core particle comprises a material selected from the group consisting of silica, biodegradable polymer, DNA-polymer polyplex, and chemotherapeutic nanocrystals.

5. The method of claim 1, wherein the core particle has a positive surface charge.

6. The method of claim 1, wherein the core particle is modified to have a positive surface charge.

7. The method of claim 6, wherein the core particle is modified with an amine group to have a positive surface charge.

8. The method of claim 1, wherein the cell membrane material comprises cell membrane fragments of cells selected from the group consisting of cancer cells, non-immune cells, and immune cells.

9. The method of claim 1, wherein the cell membrane material comprises cell membrane fragments from a cell line selected from the group consisting of CaCo-2, HepG2, MCF-7, RAW 264.7, HEK, HeLa, HITC, B16-F10, RBC, MSC.

10. The method of claim 1, wherein the core particle has a size ranging from about 50 nm to about 2 μm.

11. The method of claim 1, wherein the core particle has a surface charge ranging from about −50 mV to about +50 mV.

12. The method of claim 1, wherein the confined mixing cavity comprises a multi-inlet vortex mixer.

13. The method of claim 12, wherein a flow rate in each inlet of the multi-inlet vortex mixer ranges from about 5 mL/min to about 40 mL/min.

14. The method of claim 1, wherein a mass ratio of the cell membrane coating material to core particle ranges from about 0.1 to about 100.

15. The method of claim 1, wherein the cell membrane material comprises a tumor-associated antigen and the core particle is loaded with an adjuvant.

16. The method of claim 15, wherein the core particle comprises a mesoporous silica nanoparticle loaded with the adjuvant.

17. A biomimetic vaccine comprising the cell membrane cloaked particle prepared according to the method of claim 16.

18. A method of using flash nanocomplexation to prepare a cell membrane-cloaked particle, comprising:
loading a a cell membrane material and a core particle into a multi-inlet vortex mixer; and
turbulent mixing of the cell membrane material and the core particle in the multi-inlet vortex mixer to provide the cell membrane-cloaked particle, wherein
the turbulent mixing achieves a flow rate in each inlet of the multi-inlet vortex mixer ranging from about 5 mL/min to about 40 mL/min.

19. The method of claim 18, wherein a mass ratio of the cell membrane coating material to core particle ranges from about 0.1 to about 100.

20. The method of claim 18, wherein the core particle has a surface charge ranging from about −50 mV to about +50 mV.
